(19) United States
(12) Patent Application Publication
(10) Pub. No.: US 2009/0270640 A1
(43) Pub. Date: Oct. 29, 2009

(54) METHOD FOR PRODUCTION OF ETHYLENE OXIDE IN A MICROCHANNEL REACTOR

(75) Inventors: Torsten Maurer, Lambsheim (DE); Markus Gitter, Dresden (DE); Frank Rosowski, Mannheim (DE)

Correspondence Address: CONNOLLY BOVE LODGE & HUTZ, LLP, P O BOX 2207, WILMINGTON, DE, 19899, US

(73) Assignee: BASF AKTIENGESELLSCHAFT, Ludwigshafen (DE)

(21) Appl. No.: 12/297,895

(22) PCT Filed: Apr. 5, 2007
(86) PCT No.: PCT/EP2007/053363

§ 371 Date: Oct. 21, 2008

(30) Foreign Application Priority Data
Apr. 21, 2006 (EP) 06112890.6

(51) Int. Cl. C07D 301/04

(52) U.S. Cl. 549/523

(57) ABSTRACT

Processes for preparing ethylene oxide, the process comprising: (a) providing a catalyst-comprising microchannel reactor; (b) feeding (i) an ethylene-comprising stream and (ii) a stream comprising oxygen, an oxygen source or both, into the microchannel reactor; and (c) continuously feeding one or more components selected from the group consisting of alkyl halides, nitrogen-comprising compounds, and mixtures thereof into the microchannel reactor in a concentration of from 0.3 to 50 ppm by volume, each based on the total volume flow of all streams introduced into the reactor.

The present invention relates to an improved process for preparing ethylene oxide (EO) in a microchannel reactor, in which a stream comprising ethylene and a stream comprising oxygen or an oxygen source are fed into the microchannel reactor and conversion into ethylene oxide takes place in the catalyst-comprising microchannel reactor.

The preparation of ethylene oxide from ethylene is in principle assigned to the reaction class of epoxidations which is a subclass of oxidations. Furthermore, a distinction between these terms is not made, so that the term oxidation of ethylene is taken to mean the epoxidation of ethylene.

Various processes for preparing ethylene oxide are known and have been described. Thus, the industrial preparation of ethylene oxide by gas-phase epoxidation of ethylene by means of molecular oxygen usually takes place in externally cooled shell-and-tube reactors having tube diameters of from 20 to 50 mm and also in reactors having a loose catalyst bed and cooling tubes, for example the reactors as described in DE-A 34 14 717, EP-A 82 609 and EP-A 339 748. Here, about 10-20% of the ethylene fed into the reactor is converted into ethylene oxide and the undesirable by-product carbon dioxide. The unreacted starting materials are usually recirculated in a recycle gas (cf. Ullmann's Encyclopedia of Industrial Chemistry; 5th Ed.; Vol. A10; pp. 117-135, 123-125; VCH Verlagsgesellschaft; Weinheim 1987).

US 2006/0036106 describes the preparation of ethylene oxide by reaction in a microchannel reactor. In general, this mode of operation can be advantageous; thus, for example, improved heat removal and more intensive contact of the starting molecules (ethylene and oxygen source) are possible.

However, the known processes for preparing ethylene oxide in a microchannel reactor are in practice complicated in process engineering terms if the objective of achieving high effectiveness is to be realized. Relatively high reaction temperatures are necessary to ensure high space-time yields, but this could have an adverse effect on the selectivity to ethylene oxide. For example, a temperature range of 180-325° C. for the catalyst is disclosed in EP 266015, page 11, table 2. In addition, at high reaction temperatures in conventional reactors there is a risk that the heat of reaction produced cannot be removed to a sufficient extent. This can result in a runaway reaction in the reactor.

It is therefore an object of the invention to discover an improved process for preparing ethylene oxide in a microchannel reactor, which avoids the abovementioned disadvantages and makes it possible for the preparation of ethylene oxide to be carried out effectively and simply in process engineering terms.

We have accordingly found a process for preparing ethylene oxide in a microchannel reactor, in which an ethylene-comprising stream and a stream comprising oxygen or an oxygen source are fed into the microchannel reactor and conversion into ethylene oxide takes place in the catalyst-comprising microchannel reactor, wherein alkyl halides are fed continuously into the microchannel reactor in a concentration of from 0.3 to 50 ppm by volume, based on the total volume flow of all streams introduced into the reactor.

In an alternative embodiment, we have found a process for preparing ethylene oxide in a microchannel reactor, in which an ethylene-comprising stream and a stream comprising oxygen or an oxygen source are fed into the microchannel reactor and conversion into ethylene oxide takes place in the catalyst-comprising microchannel reactor, wherein nitrogen-comprising compounds are fed continuously into the microchannel reactor in a concentration of from 0.3 to 50 ppm by volume, based on the total volume flow of all streams introduced into the reactor.

For the present purposes, the total volume flow on which the concentrations according to the invention of alkyl halides and nitrogen-comprising compounds is based here is the total volume flow of all streams introduced into the reactor, in particular O₂, ethylene and any inert gas components comprised, e.g. N₂, methane, and any further impurities present, e.g. CO₂, CO, Ar and H₂O.

The proportion of any CO₂ present in the total stream fed into the microchannel reactor is advantageously kept low. It has been found that a CO₂ concentration of less than 2% by volume, in particular less than 1% by volume, in the microchannel reactor is particularly advantageous for the effectiveness of the process of the invention for preparing ethylene oxide by oxidation of ethylene.

In a further embodiment of the process of the invention, it is possible for both alkyl halides and nitrogen-comprising compounds to be fed in, in which case the total concentration of these two additionally introduced streams is 0.6-100 ppm by volume, based on the total volume flow of all streams introduced into the reactor, with the proportion of alkyl halides preferably being from about 0.1 to 1, particularly preferably from 0.3 to 1, based on the two streams fed in.

The targeted, continuous addition of alkyl halides and/or nitrogen-comprising compounds in the concentration range according to the invention achieves a lasting improvement in the selectivity of the catalyst. The introduction according to the invention of alkyl halides and/or nitrogen-comprising compounds reduces the formation of CO₂ by total oxidation of the ethylene. This advantageously achieves an increase in the selectivity of 0.1-10% compared to a process for the oxidation of ethylene to ethylene oxide in the microchannel reactor without introduction of alkyl halides and/or nitrogen-comprising compounds. The activity of the catalyst can also be influenced or set by means of the introduction, since a catalyst phase which is favorable for the oxidation of ethylene can be formed.

The targeted continuous introduction according to the invention of these substances in the concentration range claimed in order to improve the production process would not have been taken into consideration by a person skilled in the art for a production process in a microchannel reactor.

US 2006/0036106 merely gives a general, unelaborated indication that the feed stream could comprise an alkyl halide (page 4, paragraph 0066). A person skilled in the art will find no information with regard to positive effects which can be obtained from use in the concentration range according to the invention in the course of a targeted, continuous introduction.

EP 266015, page 11, table 2, discloses introduction of from 0.3 to 20 ppm by volume of an alkyl halide as reaction moderator. Examples mentioned in EP 266015, page 11, line 3 are 1,2-dichlorethane, vinyl chloride and chlorinated polyphenyl compounds.

The concentration range according to the invention is found to be particularly advantageous in the process for preparing ethylene oxide in a microchannel reactor. In the case of lower concentrations, there is increased formation of CO₂ by total oxidation of ethylene, which may greatly reduce the selectivity. The activity of the catalyst can also be adversely affected, since there is no formation or only delayed formation of the active phase. In the case of higher concentrations, accumulation of the alkyl halides on the catalyst can occur, e.g. as a result of excessive introduction, which leads to a reduced catalyst activity and/or selectivity through to catalyst poisoning.

The concentration of alkyl halide and/or nitrogen-comprising compound which is particularly recommended for the process of the invention depends on the specific conditions. Thus, the stream of alkyl halides or nitrogen-comprising compounds to be fed in according to the invention depends on the temperature, composition of the feed gas, type of catalysts used and the molecular structure of the alkyl halide or of the nitrogen-comprising compound.

Known microchannel reactors are generally suitable for carrying out the process of the invention. In contrast to conventional reaction apparatuses, e.g. tube/shell-and-tube or fluidized-bed reactors, microchannel reactors offer, owing to the very small dimensions of the reaction channels (dimension in at least one spatial direction of <3 mm, preferably 1 mm), inherent safety, i.e. propagation of flames or explosions is not possible (the diameter is below the minimal quench diameter). In terms of the way in which the process is carried out, there is increased freedom in terms of the choice of the organic/oxygen or air ratio, since explosion limits within the reactor do not have to be ken into account or adhered to. Design of the reactor for maximum explosion pressures is not necessary. Furthermore, short diffusion paths within the microstructures lead to greatly improved mass transfers and heat transfers which can be many times greater than those of conventional reaction apparatuses. Transport limitations which frequently occur in conventional shell-and-tube reactors are accordingly largely absent. Furthermore, the high heat removal potential of microchannel reactors makes more precise temperature control possible, so that, for example, the formation of hot spots can be suppressed and operation with an optimally selected axial temperature profile can be made possible. A runaway reaction in the reactor is effectively prevented.

Comprehensive descriptions of the configuration of microchannel reactors which in terms of their basic structure are suitable for carrying out the process of the invention may be found, for example, in US 2006/0036106 A1 and also in WO 02/18042 A1, which are hereby incorporated by reference.

For the purposes of the present invention, microchannel reactors or microreactors are reactors in general whose characteristic dimensions of the reaction channels, i.e. the dimensions in at least one spatial direction, e.g. height or width or diameter, are in the range from a few microns to a few millimeters, preferably <3 mm.

In large-scale industrial applications, too, the characteristic dimensions of the reaction space are retained. The increase in capacity is achieved by numbering-up, so that costly and time-consuming scale-up is dispensed with. The size of a production plant is thus flexible and can be inexpensively matched to requirements. Various concepts are available for introducing catalysts into microchannels (wall coatings with active materials, micro-fixed beds, metal foils, etc.).

Owing to the microeffects mentioned, microchannel reactors are in principle suitable for reactions having fast kinetics (elimination of diffusion limitations), high heat flows (improved temperature control) and substances presenting explosion hazards (runaway reactions or explosions are not possible). The use of microchannel reactors may make process intensification (higher space-time yields, product yields, selectivities) possible. As a result both capital costs (smaller, more compact apparatuses) and variable costs (raw material costs) can be reduced.

The configuration according to the invention of the process for preparing ethylene oxide using microchannel reactors enables process intensification to be advantageously achieved. This leads, inter alia, to increased productivity of the catalyst, i.e. an increased space-time yield is achieved in the microchannel reactor at a defined temperature using the same catalyst compared to conventional tube reactors.

It has been found that in the preparation of ethylene oxide under comparable process conditions, use of a microchannel reactor and an alkyl halide concentration increased to up to 50 ppm by volume compared to conventional tube reactors has a particularly advantageous effect on the selectivity and activity of the catalyst. Here, an increase in the selectivity of 0.1-5% is advantageously achieved compared to a process for the oxidation of ethylene to ethylene oxide in a microchannel reactor without increased introduction of alkyl halides.

As alkyl halides, preference is given to vinyl chloride, ethyl chloride, ethylene dichloride or mixtures thereof being fed as reaction moderators into the microchannel reactor. Particular preference is given to ethyl chloride.

An increase in the alkyl halide concentration in operation may also be advantageous for the purposes of performance optimization.

Furthermore, an introduction of 0.3-50 ppm by volume of nitrogen-comprising compounds in addition to the alkyl halides has a positive effect on the catalyst performance in the microchannel reactor. Preferred nitrogen compounds are NH₃, NO, NO₂, N₂O, N₂O₃, N₂O₃, organic nitro compounds such as nitromethane, nitroethane, 1- or 2-nitropropane. The use of NO is particularly preferred. The introduction of nitrogen-comprising compounds is carried out, in particular, in combination with nitrate or nitrite promotion, e.g. alkali metal nitrate promotion, preferably KNO₃, of the catalytically active composition.

It can also be advisable according to the invention to add only one nitrogen-comprising compound in a total concentration of from 0.3 to 50 ppm by volume, based on the total volume flow of all starting materials introduced into the reactor, in particular O₂, ethylene and any inert gas components, e.g. N₂, methane, and any further impurities present (in the recycle gas), e.g. CO₂, CO, Ar and H₂O. Here too, an increase in the selectivity of 0.1-5% compared to a process for the oxidation of ethylene to ethylene oxide in a microchannel reactor without introduction of a nitrogen-comprising compound is advantageously achieved.

Although methane can be used as inert gas in the feed gas, higher alkanes such as ethane, propane, butanes and even higher alkanes present in the feed suppress the positive effect of the alkyl halides fed in. The total concentration of higher alkanes in the feed is therefore preferably less than 5% by volume, particularly preferably less than 1% by volume. A total concentration of higher alkanes in the feed of less than 500 ppm by volume is very particularly preferred. In this context, the term “higher alkanes” refers to all saturated hydrocarbons whose empirical formula is CₙR₂ₙ₊₂ with R═H, where n≧2. The effectiveness of the process of the invention can thus be increased further by the reduction of the content of higher alkanes.

Even if the amount of alkyl halides fed in is lower or no alkyl halides at all are added, the reduction in the content of higher alkanes in the feed is found to be advantageous.

The performance improvement of the EO catalysts achieved according to the invention by introduction of alkyl halides and/or nitrogen compounds requires precise, continuous metering. Metering is usually achieved by introduction of the allyl halides and/or nitrogen compounds via the feed gas at the reactor inlet. However, the decomposition or oxidation of the alkyl halides and/or the nitrogen compounds can occur under reaction conditions, so that the effective concentration of the alkyl halides and/or nitrogen compounds metered in can vary over the length of the reactor. In addition, accumulation of the alkyl halides and/or the nitrogen compounds on the catalyst can occur as a result of, for example, excessive introduction due to an excessively high inlet concentration, and this can lead to reduced catalyst performance. The optimal concentration of the alkyl halides and/or nitrogen compounds fed in may then no longer be ensured over the entire length of the reactor.

In a particularly advantageous embodiment, the alkyl halides or the alkyl halides and/or the nitrogen compounds are therefore fed progressively into the reaction space over the length of the reactor. This specific embodiment makes very precise, stepwise introduction of the alkyl halides and/or the nitrogen compounds possible. A concentration profile over the length of the reactor which is favorable for the catalyst(s) and/or operating point(s) (concentration decreasing, constant or increasing) can thus be set and a further improved performance of the EO catalysts can be achieved.

The progressive addition can, for example, be achieved by dividing the total amount of alkyl halides and/or nitrogen compounds to be metered in into equal-sized or different-sized substreams and metering in one substteam via the feed gas at the reactor inlet and introducing at least one further substream into the reactor at a metering point, or in the case of more than two substreams at a plurality of metering points, downstream of the reactor inlet. The arrangement of the metering points for the substreams along the length of the reactor downstream of the reactor inlet is advantageously such that optimal catalyst performance, i.e. in particular a maximum selectivity, is achieved over the entire catalyst composition.

For example, the total stream can be divided into four substreams, with the reactor length L_{R} being divided into four sections, e.g. sections having a length of L_{R}/4. The first substream is metered into the first reactor section via the reactor inlet. The further three substreams are then introduced into the three reactor sections following the first reactor section after reactor lengths of L_{R}/4, 2*L_{R}/4 and 3*L_{R}/4.

In a preferred embodiment of the process of the invention, the exothermic oxidation according to the invention of ethylene to ethylene oxide in the microchannel reactor is coupled with an endothermic reaction in order to be able to utilize or remove the heat liberated in the EO synthesis. In this context, coupling means thermal coupling. Here, both the exothermic reaction for preparing ethylene oxide and the thermally coupled endothermic reaction take place in the microchannel reactor, preferably in adjacent reaction channels. As a result of these two reactions taking place within the microchannel reactor in, if appropriate, adjacent reaction channels, good heat exchange is achieved via the walls of the reaction channels, which further improves the effectiveness of the overall process. The specific configuration of such reaction channels for the coupling of exothermic and endothermic reactions in a microchannel reactor is known to those skilled in the art. Information on this subject may be found, for example, in US 2006/0036106 A1, page 16, paragraph 143. Here, it is disclosed that, in order to remove heat from the exothermic epoxidation of ethylene to form ethylene oxide, it is possible either to use a suitable and generally known heat transfer medium or to couple the reaction thermally with endothermic reactions. Examples mentioned are steam reforming reactions and dehydrogenation reactions in general. The thermal coupling is preferably achieved by means of a reforming reaction of an alcohol, since this reaction proceeds in the same temperature range as the preparation of ethylene oxide. However, the product from the reforming reaction comprises H₂ and CO, but these substances can not be utilized in the process for preparing ethylene oxide.

Furthermore, US 2006/0036106 A1, page 4, paragraph 68, proposes carrying out an oxidative dehydrogenation of ethane upstream of the preparation of ethylene oxide in a microchannel reactor, with the ethylene formed in this way being able to be passed together with an oxygen source over the EQ catalyst in order to obtain ethylene oxide. However, the preceding reaction mentioned here proves to be disadvantageous in terms of use together with the preparation according to the invention of ethylene oxide. Thus, although the ethylene obtained in the oxidative dehydrogenation of ethane can in principle be used as starting material for the preparation of ethylene oxide, the ethane which may still be present here considerably impairs, as indicated above, the positive effect of the alkyl halides fed in. An additional purification step after the oxidative dehydrogenation of ethane is therefore necessary.

In a preferred embodiment of the process of the invention, the exothermic preparation of ethylene oxide is thermally coupled in the manner indicated above with the endothermic reaction of the dehydration of ethanol. This is found to be particularly advantageous since ethylene can here be obtained as product in very high yields. A further advantage is that the ethylene formed can be fed to the ethylene oxide synthesis. The water formed in the dehydration and/or other products formed are preferably separated off from the resulting ethylene by, for example, condensation and the ethylene is then fed to the ethylene oxide synthesis.

Ethylene can generally be prepared by steam cracking of oil or naphtha or by steam cracking of ethane. Ethylene can also be prepared by catalytic, oxidative or autothermal dehydrogenation of ethane. Further processors for preparing ethylene are the oxidative coupling of methane or metathesis reactions of higher olefins such as propene. A substantial disadvantage of all these processes is the dependence on fossil raw materials such as oil and natural gas.

However, apart from the processes mentioned, ethylene can also be prepared by catalytic dehydration of ethanol. The catalytic dehydration of ethanol is an endothermic reaction. As catalysts, it is possible to use oxidic catalysts (e.g. Al₂O₃, ZrO₂ (Bull. Soc. Chem. Jpn. 1975, 48, 3377), salts (sulfates (J. Catal. 1971, 22, 23), phosphates (Kinet. Katal. 1964, 5, 347), (hetero)polyphosphoric acids (Chem. Lett. 1981, 391.; Ind. Eng. Chem., Prod. Res. Dev. 1981, 20, 734 (S: >97%, Y: >90%, T: <300° C.)), ion exchange resins or supported mineral acids in the temperature range up to 400° C. Particularly preferred catalysts for the dehydration of ethanol are zeolites which can be used in the temperature range 200-300° C. (e.g. ZSM-5 (J. Catal. 1978, 53, 40), selectivity: 98%, conversion: 100%).

The synthesis of EO over silver catalysts usually takes place in the temperature range 200-300° C. It is therefore a particularly advantageous embodiment of the process of the invention to couple the exothermic synthesis of ethylene oxide from ethylene in a microchannel reactor with an endothermic, catalytic dehydration of ethanol to ethylene. Here, the term “couple” once again refers to the above-described thermal coupling in preferably adjacent microchannels.

It can also be found to be advantageous in general to couple the exothermic oxidation reaction of ethylene to ethylene oxide with an endothermic reaction even in the case of the preparation of ethylene oxide in a microchannel reactor without the continuous addition according to the invention of alkyl halides or nitrogen-comprising compounds.

As catalysts in microchannel reactors, it is possible to use all silver-comprising catalysts, if appropriate on a suitable support material, which are generally suitable for the preparation of ethylene oxide from ethylene and oxygen. Examples of generally customary promoter-doped silver catalysts which are suitable for our process are, for example, the silver catalysts of DE-A 23 00 512, DE-A 25 21 906, EP-A 14 457, DE-A 24 54 972, EP-A 172 565, EP-A 357 293, EP-A 11 356, EP A 85 237, DE-A 25 60 684, DE-A 27 53 359 and EP 266015.

Particularly suitable promoters for EO catalysts are the elements nitrogen, sulfur, phosphorus, boron, fluorine, group IA metals, group IIA metals, rhenium, molybdenum, tungsten, chromium, nickel, copper, platinum, palladium, titanium, hafnium, zirconium, vanadium, thallium, thorium, tantalum, niobium, gallium, indium, tin and germanium and also mixtures thereof.

To give a better indication of what type of catalysts can be used in the process of the invention, mention may be made by way of example of silver catalysts having a silver content of from 5 to 50% by weight, in particular from 6 to 30% by weight, based on the total catalyst composition, a content of the light alkali metals lithium and/or sodium of from 1 to 5000 ppm by weight, a content of the heavy alkali metals rubidium and/or cesium of from 1 to 5000 ppm by weight, a tungsten content of from 1 to 5000 ppm by weight, a molybdenum content of from 1 to 3000 ppm by weight and/or a rhenium content of from 1 to 10 000 ppm by weight and also a content of sulfur and/or phosphorus and/or boron of from 1 to 3000 ppm by weight, based on the total catalyst composition.

As support material, it is in principle possible to use any porous material which is stable under the conditions of the ethylene oxide synthesis, for example activated carbon, aluminum oxides, titanium dioxide, zirconium dioxide or silicon dioxide or other ceramic compositions or corresponding mixtures.

Silver can likewise be used in the form of, for example, a foil or a mesh or a felt as catalyst in the microchannel reactor.

The process of the invention offers an effective and technically simple way of preparing ethylene oxide in a microchannel reactor. As a result of the targeted, continuous addition of alkyl halides and/or nitrogen-comprising compounds in the range claimed, a particularly large increase in the effectiveness is achieved. These advantages are increased further in the case of progressive addition.

1-12. (canceled)

13. A process for preparing ethylene oxide, the process comprising:
(a) providing a catalyst-comprising microchannel reactor;
(b) feeding (i) an ethylene-comprising stream and (ii) a stream comprising oxygen, an oxygen source or both, into the microchannel reactor; and
(c) continuously feeding one or more components selected from the group consisting of alkyl halides, nitrogen-comprising compounds, and mixtures thereof into the microchannel reactor in a concentration of from 0.3 to 50 ppm by volume, each based on the total volume flow of all streams introduced into the reactor.

14. The process according to claim 13, wherein the one or more components comprises an alkyl halide.

15. The process according to claim 14, wherein the microchannel reactor comprises a reaction channel having a dimension in at least one spatial direction of <1 mm.

16. The process according to claim 13, wherein the one or more components comprises a nitrogen-comprising compound.

17. The process according to claim 13, wherein the one or more components comprises an alkyl halide and a nitrogen-comprising compound.

18. The process according to claim 13, wherein the microchannel reactor has a reaction space and a length, and wherein the one or more components are fed in progressively into the reaction space over the length of the microchannel reactor.

19. The process according to claim 15, wherein the microchannel reactor has a reaction space and a length, and wherein the one or more components are fed in progressively into the reaction space over the length of the microchannel reactor.

20. The process according to claim 16, wherein the microchannel reactor has a reaction space and a length, and wherein the one or more components are fed in progressively into the reaction space over the length of the microchannel reactor.

21. The process according to claim 17, wherein the microchannel reactor has a reaction space and a length, and wherein the one or more components are fed in progressively into the reaction space over the length of the microchannel reactor.

22. The process according to claim 13, wherein one or more of the streams fed into the microchannel reactor is subjected to a higher alkane content reduction such that the one or more streams has a higher alkane content below 5% by volume prior to being fed into the microchannel reactor.

23. The process according to claim 18, wherein one or more of the streams fed into the microchannel reactor is subjected to a higher alkane content reduction such that the one or more streams has a higher alkane content below 5% by volume prior to being fed into the microchannel reactor.

24. The process according to claim 14, wherein the alkyl halide comprises ethyl chloride.

25. The process according to claim 17, wherein the alkyl halide comprises ethyl chloride.

26. The process according to claim 16, wherein the nitrogen-comprising compound comprises NO.

27. The process according to claim 17, wherein the nitrogen-comprising compound comprises NO.

28. The process according to claim 13, wherein the reaction of the ethylene-comprising stream and the stream comprising oxygen, an oxygen source or both to prepare ethylene oxide is coupled with an endothermic reaction.

29. The process according to claim 28, wherein the endothermic reaction comprises a catalytic dehydration of ethanol to ethylene.

30. The process according to claim 13, wherein the microchannel catalyst comprises silver and at least one additional element, wherein the additional element is selected from the group consisting of nitrogen, sulfur, phosphorus, boron, fluorine, Group IA metals, Group IIA metals, rhenium, molybdenum, tungsten, chromium, nickel, copper, platinum, palladium, titanium, hafnium, zirconium, vanadium, thallium, thorium, tantalum, niobium, gallium, indium, tin, germanium, mixtures thereof, and compounds thereof, and wherein the catalyst is present in a manner selected from the group consisting of (i) on a support material, (ii) applied to one or more walls of the microchannel reactor, (iii) applied to an intermediate layer of an oxidic material disposed on one or more walls of the microchannel reactor, and combinations thereof.

31. The process according to claim 13, wherein the microchannel catalyst comprises silver, rhenium or a compound thereof, and at least one additional element, wherein the additional element is selected from the group consisting of nitrogen, sulfur, phosphorus, boron, fluorine, Group IA metals, Group IIA metals, molybdenum, tungsten, chromium, nickel, copper, platinum, palladium, titanium, hafnium, zirconium, vanadium, thallium, thorium, tantalum, niobium, gallium, indium, tin, germanium, mixtures thereof, and compounds thereof, and wherein the catalyst is present in a manner selected from the group consisting of (i) on a support material, (ii) applied to one or more walls of the microchannel reactor, (iii) applied to an intermediate layer of an oxidic material disposed on one or more walls of the microchannel reactor, and combinations thereof.

32. The process according to claim 13, wherein the process is carried out at a CO₂ concentration in the total volume of streams fed into the microchannel reactor of less than 2% by volume.
